(19) United States
(12) Patent Application Publication
(10) Pub. No.: US 2005/0181367 A1
(43) Pub. Date: Aug. 18, 2005

(54) Method of concentrating macromolecules or agglomerates of molecules or particles

(75) Inventors: Jean Berthier, Meylan (FR); Laurent Davoust, Grenoble (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: COMMISSARIAT A L'ENERGIE ATOMIQUE, Paris 15eme (FR); Centre National De La Recherche Scient., Paris Cedex 16 (FR)

(21) Appl. No.: 10/507,521

(22) PCT Filed: Mar. 24, 2003
(86) PCT No.: PCT/FR03/00920

(30) Foreign Application Priority Data
Mar. 25, 2002 (FR) 02/3690

(51) Int. Cl. C12Q 1/68; C12P 19/34; C12N 1/08

(52) U.S. Cl. 435/6; 435/91.2; 435/270

(57) ABSTRACT

A subject of the present invention is a method for the selective concentration of a macromolecule or of an agglomerate of molecules of particles contained in a liquid sample, comprising successively the following steps: formation of a stabilized dispersion of foam or emulsion type, from a medium comprising said liquid sample and an interface layer, said interface layer being capable of selectively fixing said macromolecule or said agglomerate to be concentrated; and resorption of the dispersion formed during the preceding step so as to reform said interface layer. The present invention can be applied to the concentration of DNA, of proteins, of prions, of colloidal particles.

### TECHNICAL FIELD

The present invention relates to a method for the concentration of macromolecules or of agglomerates of molecules or of particles, for the purpose of possibly detecting or specifically extracting said macromolecules or said agglomerates.

### STATE OF THE PRIOR ART

Currently, the techniques of the concentration of macromolecules or of agglomerates of molecules are, for the most part, intended for the field of medical diagnosis, in particular for the field of the detection of DNA strands or of protein complexes such as antigens or prions.

For the detection of DNA strands, the current technique consists, most commonly, in concentrating the DNA strands by amplification in a liquid medium, according to the technique commonly referred to by the abbreviation PCR (“Polymerase Chain Reaction”).

This technique consists in replicating the DNA strands contained in a liquid sample a large number of times (up to 10⁵-10⁶ times) by injecting polymerase into the sample subjected to a series of thermal cycles.

After quite a large number of thermal cycles, in any case more than 10, the concentration of DNA is sufficiently high to allow detection.

However, this technique, as explained above, requires a considerable period of time, due to the significant number of thermal cycles to be reproduced in order to have a sufficient amount of DNA. In addition, this technique is accompanied by a considerable background noise due to the fact that the polymerase can amplify DNA segments present in the liquid sample which are different in nature from the DNA segments to be detected.

In order to overcome these disadvantages, alternative methods to PCR have been developed. Among these methods, mention may be made of a method not requiring amplification. The principle of this method of detection without amplification is based on the capture of the target DNA segments, as few as they are. It consists in hybridizing the target DNA segments with functionalized paramagnetic nanobeads so as to concentrate said segments at the surface of these beads before detection. However, this method comes up against the problem of non-specific adsorption; some paramagnetic beads, coated with latex, attach to the solid wall of the reactor, where the method is carried out, under the effect of hydrophobicity or of electrical forces. The sensitivity achieved is then no longer that expected.

As regards the detection of proteins such as antibodies or antigens, two types of tests are distinguished: “homogeneous phase” tests and “heterogeneous phase” tests. The homogeneous phase tests take place in solution. The amount of antibodies used is measured directly in the solution by attachment of this antibody to the complementary antigen, without there being any physical separation between the free antibodies and the antibodies bound to an antigen. This distinction between free antibody and bound antibody is made in various ways, in particular based, for example, on a transfer of fluorescence between two antibodies (the antigen being sandwiched between two labelled antibodies for example). These tests have the advantage of being rapid and relatively inexpensive. It is these which are used massively in high throughput screening programmes in the pharmaceutical industry for example. However, their sensitivity is limited, to around 1 nM, as regards the tests based on nucleic acids. For this reason, the diagnostic industry preferentially uses heterogeneous phase tests, in which an antibody is immobilized on a solid support, which support can then be washed in order to remove virtually all the labelling reagents. In addition, an amplification of the signal is carried out, using an enzyme as labelling molecule. These tests on a solid support are slower and more expensive than those in homogeneous phase, but they make it possible to achieve much higher sensitivities, in the region of 0.1 pM, i.e. 10 000 times better than the homogeneous phase tests. However, the use of a solid support makes these tests difficult to carry out.

As regards protein complexes such as prions, they must also, in order to be detectable in physiological liquids such as blood, undergo a concentration phase. For these molecules, the PCR technique described above cannot be used because they do not contain nucleotides. As a result, research studies relating to the prion field have recently resulted in the setting up of a method referred to as “protein misfolding cyclic amplification” or PMCA.

Prions, which are responsible in particular for spongiform encephalopathy, are complexes or agglomerates consisting of a natural protein, the PrP^{C} glycoprotein, normally present at the surface of many cells in the organism and of an infectious protein PrP^{SC}, which differs from a normal glycoprotein PrP^{C} only by virtue of its conformation, which is misfolded. The PrP^{SC} proteins are, firstly, capable of combining with the PrP^{C} proteins and, secondly, capable of inducing the conversion of normal proteins into infectious proteins. The detection of prions is made difficult by the fact that they are present in notable amount only in the brain, whereas they are present in trace amounts in the blood.

The PMCA technique thus makes possible to concentrate the prions in the sample medium, i.e. the blood, so that they can be detected.

To do this, a method of sonication intended to fragment the prions is used. All the prions derived from this ultrasound fragmentation regrow in vitro with the aid of the PrP^{C} proteins of the sample. This elementary cycle (fragmentation-regrowth) is reproduced as many times as is necessary, until the amount of prions becomes detectable.

The detection step, facilitated by the amplification step, is carried out by fluorescence spectroscopy. The proteins to be detected are labelled with a fluorescence probe, the presence of which manifests itself when said probe is illuminated with a light of characteristic wavelength. However, this technology most commonly engenders a not insignificant background noise due to the fact that the probes can combine with molecules other than the prions to be detected.

A more developed technique, referred to as fluorescence correlation technique, uses two fluorescent probes which emit according to two different wavelengths and which both attach symmetrically and specifically to the PrP^{C} proteins, the latter being present at a considerable concentration with the prions. A very small volume of the liquid containing the prions to be detected is then illuminated with beams at two different wavelengths and the two intense fluorescent emissions due to the presence of prions are detected in a non-steady manner, which prions constitute an agglomerate of normal or infectious PrP proteins to which the fluorescent probes are attached. However, a background noise remains, due in particular to the presence of fluorescent probes which have not been able to find specific adsorption sites, and to the presence of isolated normal PrP^{C} proteins to which fluorescent probes have attached.

Thus, the methods of concentration disclosed above all exhibit one or more of the following disadvantages:
they do not allow specific concentration of the macromolecules or agglomerates to be concentrated, due to the fact that some of these techniques can generate species which can result, in particular during possible detection, in a considerable background noise;
they do not allow a sufficient concentration of the macromolecules or agglomerates for the purpose of a possible detection.

### DISCLOSURE OF THE INVENTION

The subject of the present invention is precisely to propose a method for the concentration of macromolecules or of agglomerates of molecules or of particles, which in particular allows a selective concentration of said macromolecules or said agglomerates, for the purpose of a possible detection, by limiting as much as possible the background noises, or for the purpose of a possible purification of a sample containing said macromolecules or said agglomerates.

To do this, a subject of the present invention is a method for the selective concentration of a macromolecule or of an agglomerate of molecules or of particles contained in a liquid sample, comprising successively the following steps:
formation of a stabilized dispersion of foam or emulsion type, from a medium comprising said liquid sample and an interface layer, said interface layer being capable of selectively fixing said macromolecule or said agglomerate to be concentrated; and
resorption of the dispersion formed during the preceding step so as to reform said interface layer.

The term “interface layer” is intended to mean, according to the invention, a monolayer (or virtually two-dimensional zone) located at the surface of the liquid sample (referred to as first liquid phase) comprising the macromolecule or the agglomerate to be concentrated. This layer, by virtue of its nature and specific properties, is able to provide the selective transfer of the macromolecule or of the agglomerate from the liquid sample to the interface layer and, due to its tiny volume compared to the liquid sample, of concentrating said macromolecule or said agglomerate.

Depending on the nature of the macromolecule or of the agglomerate to be concentrated, the interface layer may correspond to a second liquid phase deposited at the surface of the liquid sample (the case, for example, when it is a question of concentrating a macromolecule of the DNA type), this phase having characteristics such that it makes it possible to attract the macromolecule or the agglomerate towards it. The interface layer may also correspond to the interface between the ambient atmosphere and the liquid sample (for example, when the macromolecule or the agglomerate to be concentrated is hydrophobic in nature, such as prions, and when the liquid sample containing it is an aqueous medium).

When the interface layer corresponds to a second liquid phase, the method of the invention may comprise a prior step (located before the dispersion formation step) consisting in depositing said second liquid phase or interface layer at the surface of the liquid sample.

Generally, the dispersion formation step is carried out by mechanical agitation of the medium comprising the liquid sample and the interface layer or by injection, directly into the liquid sample surmounted by the interface layer of gaseous or liquid capillary jets.

It is noted that a foam denotes a dispersion comprising a combination of bubbles of gas (typically air) coexisting with an interstitial liquid medium, in the form of thin interstitial films between the bubbles.

This type of dispersion thus brings about a multitude of liquid-gas interfaces. The foam according to the invention can be obtained, for example, by vigorous mechanical agitation of the abovementioned medium or by injection into this medium of capillary jets of gas (typically jets of air).

It is noted that, according to the invention, an emulsion denotes a dispersion in which the interface layer is divided up into globules within the liquid sample, said liquid sample constituting an interstitial medium. A multitude of liquid-liquid interfaces is thus formed, and the contact surface between the liquid sample and the interface layer is as a result considerably increased.

The emulsion, just as the foam, can be obtained by mechanical agitation of the medium comprising the liquid sample and the interface layer, but also by direct injection into the liquid sample surmounted by the interface layer of liquid or gaseous capillary jets.

Thus, according to the invention, the fact that the procedure involves a dispersion of the foam or emulsion type for concentrating a macromolecule or an agglomerate of molecules or particles contributes to bringing about a multitude of interstitial zones between the liquid sample and the interface layer, which considerably increases the amount of surface between these two media and as a result facilitates the fixing of the macromolecules or the agglomerates of molecules by the dispersed interface layer. This fixing takes place in virtually two-dimensional zones, due to the fact that it occurs in the interstitial zones, which greatly improves the efficiency and the time for capture of the macromolecules to be concentrated by the interface layer.

If the structure of the functionalized interface layer is potentially unstable under the effect of the creation of interface air, advantage can be taken of the Rayleigh instability to produce an emulsion under virtually static conditions, the most dense liquid being in a position above the least dense liquid under the initial conditions.

After resorption of the dispersion, there is thus once again a reformed interface layer containing a concentration of macromolecules or agglomerates of molecules, and a liquid phase corresponding to the liquid sample completely or partly devoid of said macromolecules or of said agglomerates.

The advantage of the present invention is therefore to be able to rapidly, and without amplification, concentrate the macromolecules or agglomerates selectively, by involving the formation of a dispersion which increases the efficiency of the concentration.

As described above, after capture of the macromolecules or agglomerate of molecules or particles to be concentrated, the dispersion is made to undergo a resorption step. This step can be carried out in various ways. For example, this dispersion resorption step can be carried out by drainage of the interstitial films, in the case of a foam, or of the interstitial medium, in the case of an emulsion. The resorption kinetics can be controlled either by a judicious choice, where appropriate, of the molecules making up the interface layer, of the macromolecules or agglomerates via the length of the molecular chains, for example, by means of mechanical shearing of the dispersion.

According to the nature of the interface layer and of the macromolecule or of the agglomerate to be concentrated, and in particular when the interface layer corresponds to a second liquid phase, the interface layer may comprise at least one molecule capable of selectively fixing the macromolecules or the agglomerates of molecules or particles in question.

According to this embodiment, the molecule capable of fixing the macromolecule or agglomerate of molecules or particles to be concentrated is contained in the interface layer from the start, before formation of the foam or of the emulsion; it may be a molecule comprising groups capable of fixing the macromolecule or said agglomerate by chemical affinity, electric or magnetic polarization, and/or ionization, it being possible for said molecule to preferably be a surfactant molecule.

If said molecule is not mixed with other surfactant molecules, said molecule may also be, due to surfactant properties, a molecule for stabilizing the dispersion formed in the course of one of the steps of the method.

In this case, the molecule performs a double function, namely to fix the macromolecule or the agglomerate to be concentrated and also to stabilize the dispersion, thus contributing to increasing the contact time in the interstitial zones between attractive molecules and macromolecules or agglomerates to be concentrated.

The method according to the invention may be applied to the concentration of any type of macromolecules or agglomerates.

By way of examples, mention may be made, as macromolecules which can be concentrated, according to the method of the invention, of nucleic acids, or proteins such as antibodies or antigens.

By way of examples, mention may be made, as agglomerates of molecules which can be concentrated, according to the method of the invention, of prions.

By way of examples, mention may be made, as agglomerates of particles which can be concentrated, of colloidal particles such as gold particles.

Thus, the method according to the invention can be used for concentrating a particular nucleic acid, which is DNA.

In the case of DNA, the interface layer corresponds to a second liquid phase comprising a molecule capable of fixing the DNA, which is, for example, a molecule functionalized with a probe (such as a DNA complementary to the DNA to be concentrated) so as to allow the specific hybridization of the DNA to be concentrated, for example a lipid functionalized with a DNA complementary to the DNA to be concentrated.

The fact that the molecule is a lipid is particularly advantageous in the context of this invention, since this category of molecules contributes to providing the stabilization of the dispersion formed. In addition, the functionalization of this molecule with a DNA complementary to the DNA to be concentrated allows selective concentration and extraction of said DNA.

By way of examples, mention may be made, as functionalized lipids that are effective in concentrating DNA, of biotinylated lipids comprising an avidin group or avidin derivative, onto which the complementary DNA is grafted by means of a biotinylated end incorporated into said complementary DNA beforehand, or else cationic lipids comprising at least one spermine group onto which the complementary DNA is adsorbed. Such cationic lipids may be lipids of the DOGS or dioctadecylamidoglycylspermine type, sold under the trade name Transfectam™. These lipids have two saturated C₁₈ carbon chains and a polar head consisting of a spermine group exhibiting high affinity for DNA. The complementary DNA is thus adsorbed onto the spermine sites. The resulting lipids constitute real functionalized probes for the specific hybridization of the DNAs to be concentrated, referred to as “target DNAs”.

The method according to the invention can be used for the selective concentration of antigens or of antibodies contained in a liquid sample, without involving a solid support, as is the case of the implementations of the prior art. According to this embodiment, the interface layer corresponds to a second liquid phase deposited at the surface of the liquid sample and comprises carriers or liposomes consisting of phospholipids and containing the antibody or antigen complementary to the antigen or the antibody to be concentrated.

The method according to the invention can also be used for the concentration of agglomerates of molecules, such as prions. In the case of prions, the interface layer corresponds to the (liquid sample/ambient atmosphere) interface, the interface layer being able to selectively concentrate the prions due to the hydrophobic nature of the prions (the liquid sample being an aqueous medium such as blood).

The method according to the invention can also be used for the concentration of colloidal particles. These particles may be, for example, submicronic particles of gold solubilized in water (corresponding, according to the terminology of the invention, to the first liquid phase). In this case, the interface layer corresponds to a second liquid phase comprising molecules capable of fixing these colloidal particles of gold, said molecules being, for example, molecules bearing thiol—SH groups.

The concentration method, explained above, serves, as its name indicates, to selectively concentrate, in an interface layer, a given macromolecule or agglomerate of macromolecules. It can, as a result, be used in order to purify, detect or amplify the given macromolecule or a given agglomerate of molecules or particles.

Thus, a subject of the present invention is also a method for the purification of a macromolecule or of an agglomerate of molecules or particles initially contained in a liquid sample, comprising the concentration of said macromolecule or of said agglomerate within said interface layer using the concentration method described above, followed by the elimination of the liquid sample depleted of said macromolecule or said agglomerate, after the concentration step.

For example, there is an application for this method in the case of DNA purification. In this case, the method according to the invention consists, using molecules functionalized with a specific complementary DNA, in specifically extracting a target DNA from a liquid sample comprising, for example, a mixture of various DNAs, or various portions of DNA, said sample then being eliminated.

There can also be an application for this method in protein purification. The selective capture of proteins via the layer of functionalized lipids, followed by a subsequent step of crystallization of said proteins, can make it possible to isolate these proteins in order to study the structure thereof, or else make it possible to purify a solution of the protein in question.

A subject of the present invention is also a method for the detection of a macromolecule or of an agglomerate of molecules or particles initially contained in a liquid sample, comprising the concentration, within an interface layer, of said macromolecule or of said agglomerate using the concentration method described above, followed by the detection of said macromolecule or of said agglomerate within said layer by means of appropriate detection techniques.

Thus, when the macromolecule is DNA, the detection of the DNA, after selective concentration, can be carried out by laser-excited fluorescence or by detecting the variation of the surface electric potential within the functionalized layer, or else by an interfacial rheology technique. The performance levels of the fluorescence detection or electrical detection can in particular be improved by compressing, by means of a mechanical or hydrodynamic method, the interface layer containing the hybridized target DNAs at a point of the interface (at the centre, for example) coinciding with the laser excitation volume or else with the presence of an electric probe.

Finally, a subject of the present invention is also a method for the amplification of a macromolecule or of an agglomerate of molecules or of particles initially contained in a liquid sample, comprising the concentration of said macromolecule or of said agglomerate within an interface layer using the concentration method described above, the replacement of said liquid sample, after the step for concentrating said macromolecule or said agglomerate within said layer, with a liquid comprising amplification agents, followed by the step of amplification by means of said agents.

Thus, when the amplification method applies to DNA, after concentration of the target DNA segments in the interface layer, the liquid sample depleted of these segments is withdrawn and replaced with purified liquid containing amplification agents such as polymerase and deoxyribonucleotides. The PCR amplification phase can then be carried out without the presence of parasite DNA segments and the background noise for the PCR is consequently considerably reduced.

For example, when the amplification method applies to agglomerates of molecules such as prions, after concentration of the prions in a given interface layer, according to the concentration method described above, the liquid sample depleted of said prions can be withdrawn and replaced with a pure liquid containing amplification agents such as normal PrP^{C} proteins which have not yet been transformed. Next, the conventional steps of the PMCA (sonication, etc.) can be carried out without being impaired by parasite molecules.

This amplification method can be followed by an ultra-sensitive detection, for example by fluorescence correlation, which may or may not be carried out jointly with the PMCA. By means of this technique, it is possible to improve the performance levels of the detection by concentrating, using a mechanical or hydrodynamic method, the prions locally at a point of the interface layer which coincides with the laser measuring volume or else with the presence of an electric probe.

Other characteristics and advantages of the invention will emerge more fully on reading the description of the particular embodiment which follows, with reference to the attached drawings.

### BRIEF DESCRIPTION OF THE FIGURES

FIG. 1 represents the various steps for achieving the concentration of a target DNA according to a particular embodiment of the concentration method according to the invention.

FIG. 2 represents a detailed view of an interstitial film created during the formation of a foam.

FIGS. 3, 4 and 5 represent methods of purification, detection and concentration carried out after the concentration method explained in FIG. 1.

### DISCLOSURE OF A PARTICULAR EMBODIMENT OF THE INVENTION

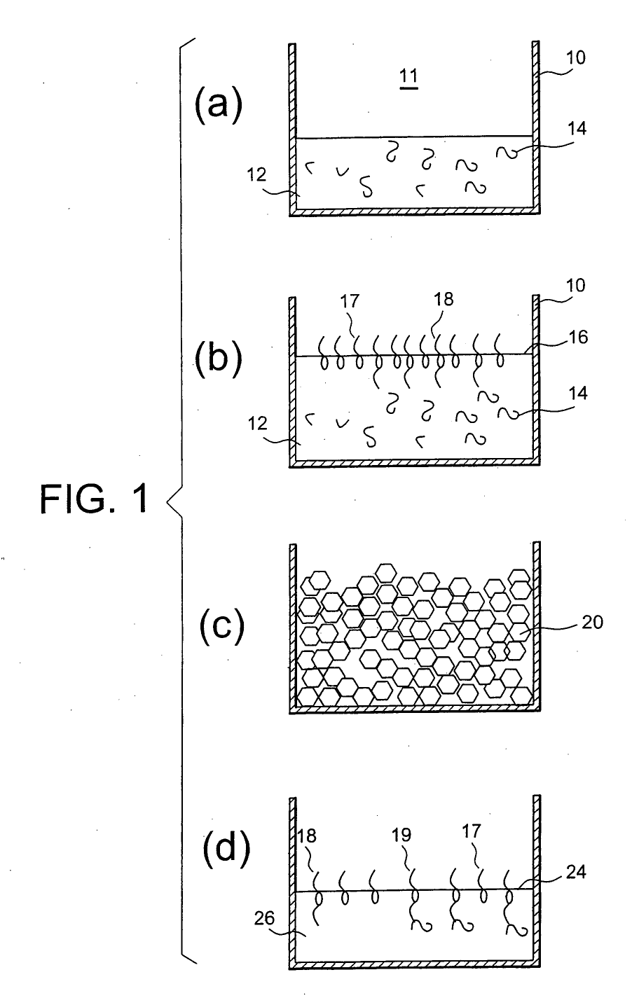

FIG. 1 represents, by way of nonlimiting illustration, the implementation, in four steps (a), (b), (c) and (d), of the method according to the invention for the concentration of a target DNA.

Step (a) of FIG. 1 represents a microbeaker 10 into which is placed, using a micropipette or syringe, a liquid sample 12 containing the target DNA 14, represented in the figure in the form of strands. Said sample 12 is surmounted by the ambient atmosphere 11.

In the course of the step entitled (b) in FIG. 1, a monolayer 16 comprising a mixture of ligand lipids 18 and of diluting lipids 17 (ligand lipids/diluting lipids ratio of 1:4) is deposited at the surface of the liquid sample 12. This monolayer 16 corresponds, according to the terminology of the invention, to an interface layer. These ligand lipids 18 are such that the target DNAs will be able to hybridize with said lipids. For this, these lipids must be functionalized during a preliminary step. Thus, the ligand lipids can be lipids that are initially biotinylated, of the biotin-(LC)-DPPE type, onto which, firstly, avidin is adsorbed and then, secondly, the DNA complementary to the target DNA is grafted onto the avidin by means of a biotin group attached to one of the ends of the complementary DNA. The combination (biotinylated lipid-avidin-biotinylated complementary DNA) constitutes a lipid functionalized with a probe for specific hybridization of the target DNA strands.

The lipids may also be cationic lipids comprising at least one spermine end, onto which a DNA complementary to the target DNA to be concentrated is adsorbed.

It is clearly understood that the molecules capable of attaching the target DNA can extend to any type of molecules able to selectively fix the target DNA.

In accordance with the invention and as illustrated by step (c) in FIG. 1, a dispersion 20, of the foam type, is created by injecting air into the liquid sample, said foam consisting of a set of bubbles kept in cohesion by interstitial liquid films. The temporary stability of the foam is provided by the lipids making up the phase in the form of a monolayer 16.

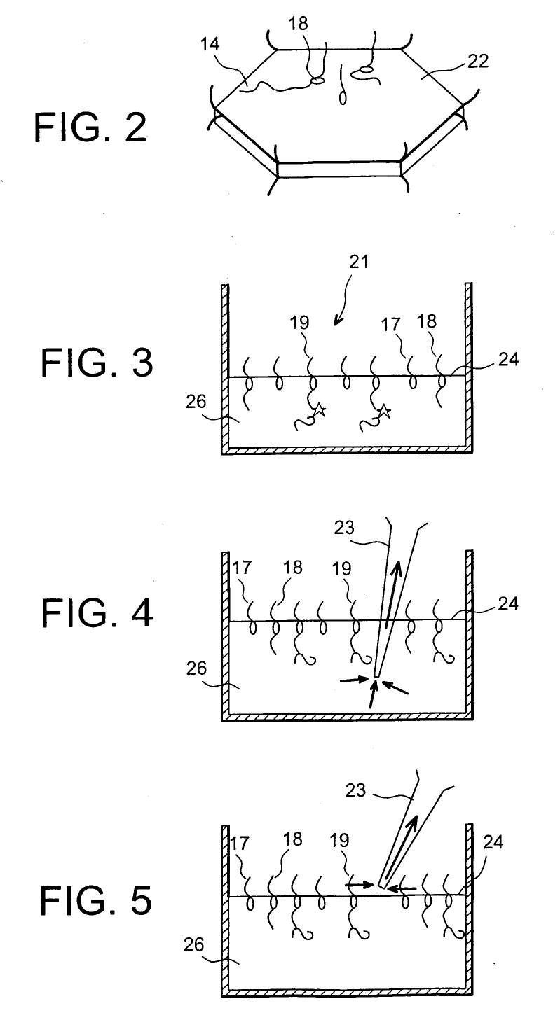

A detailed view of an interstitial liquid film making up the dispersion of foam type is represented in FIG. 2. In this figure, this film has an octahedral shape 22 with a very small volume, within which the liquid sample 12 and the monolayer 16 or interface layer coexist. As a result of this, the target DNA 14 is virtually in direct contact with the functionalized lipids 18 of the monolayer or interface layer and is thus very rapidly adsorbed by these lipids 18. The involvement of a foam multiplies the efficiency and the kinetics of capture of the DNA by the lipids, due to the very small volume of the interstitial films making up the foam.

Finally, in the course of an ultimate step represented in (d) in FIG. 1, the foam is resorbed, leaving place again for a nondispersed two-phase medium comprising the lipid monolayer 24, which is very thin, at the level of which the target DNAs have adsorbed onto the hybridized lipids 19, this monolayer corresponding to an interface layer given as reference 24 on the drawing and a phase 26 corresponding to the liquid sample 12 depleted of target DNA. It is clearly understood that, throughout FIG. 1, the lipids 17, 18 and 19 should only be located solely at the level of the monolayer 16 or interface layer 24 (for FIG. 1d), of minute thickness. However, for reasons of visibility, said lipids have been considerably enlarged.

FIGS. 3, 4 and 5 illustrate various applications of the concentration method that can be envisaged, implemented according to a particular embodiment described above.

Thus, FIG. 3 illustrates the case where, after concentration of said target DNAs, by the method explained in FIG. 1, said DNAs are detected by fluorescence techniques. In this case, the lipids 19 functionalized with a DNA complementary to the target DNA and hybridized comprise, in addition, a fluorescence label. Thus, it is possible to determine the presence of target DNA at the interface layer 24 by means of a fluorescence measurement.

FIG. 4 illustrates a particular case of purification of a DNA concentrated beforehand, by means of the concentration method explained according to FIG. 1. Once the resorption of the foam has finished, the phase 26 depleted of DNA, said DNA being adsorbed for the most part on the layer 24, is withdrawn by means of a micropipette 23, and an interface layer 24 of purified DNA is obtained.

FIG. 5 illustrates the case where the concentration method is used with the sole aim of obtaining a phase with a higher concentration of a given DNA than the original phase of said DNA. At this time, the layer 24 rich in target DNA is withdrawn by means of a micropipette 23 in order to be used for various applications.

It is clearly understood that other applications, not represented in these figures, can be envisaged.

1. A method for the selective concentration of a macromolecule or of an agglomerate of molecules or of particles comprised in a liquid sample, the method successively comprising:
forming a stabilized dispersion of foam or emulsion type, from a medium comprising said liquid sample and an interface layer, said interface layer being capable of selectively fixing said macromolecule or said agglomerate to be concentrated; and
reforming said interface layer by the resorption of the dispersion formed during said forming a stabilized dispersion.

2. The method according to claim 1, wherein said forming a stabilized dispersion the dispersion formation step is carried out by mechanical agitation of the medium comprising said liquid sample and said interface layer.

3. The method according to claim 1, wherein said forming a stabilized dispersion is carried out by injection, directly into the liquid sample, of gaseous or liquid capillary jets.

4. The method according to claim 1, wherein the interface layer comprises at least one molecule capable of selectively fixing said macromolecule or said agglomerate.

5. The method according to claim 4, wherein the molecule capable of fixing the macromolecule or agglomerate of molecules or of particles to be concentrated is a molecule comprising groups capable of fixing the macromolecule or agglomerate by chemical affinity, electric or magnetic polarization, and/or ionization.

6. The method according to claim 1, wherein the macromolecule is selected from the group consisting of nucleic acids, proteins, antigens and antibodies.

7. The method according to claim 1, wherein the agglomerate of molecules is a prion.

8. The method according to claim 1, wherein the agglomerate of particles is colloidal particles.

9. The method according to claim 1, wherein the macromolecule to be concentrated is DNA.

10. The method according to claim 4, wherein the macromolecule to be concentrated is DNA, and the molecule capable of fixing the DNA is functionalized with a probe to allow the specific hybridization of the DNA to be concentrated.

11. The method according to claim 10, wherein the molecule capable of fixing the DNA is a lipid functionalized with a DNA probe complementary to the DNA to be concentrated.

12. The method according to claim 11, wherein the lipid is a biotinylated lipid comprising an avidin group or avidin derivative, onto which the complementary DNA is grafted by means of a biotinylated end incorporated into said DNA beforehand.

13. The method according to claim 11, wherein the lipid is a cationic lipid comprising at least one spermine group onto which the complementary DNA is adsorbed.

14. A method for the purification of a macromolecule or of an agglomerate of molecules or particles initially comprised in a liquid sample, the method comprising
concentrating said macromolecule or said agglomerate within a layer using the method according to claim 1, and then
eliminating the liquid sample depleted of said macromolecule or said agglomerate.

15. A method for the detection of a macromolecule or of an agglomerate of molecules or particles initially comprised in a liquid sample, the method comprising
concentrating, within a layer, said macromolecule or said agglomerate using the method according to claim 1, and
detecting said macromolecule or said agglomerate within said layer.

16. A method for the amplification of a macromolecule or of an agglomerate of molecules or of particles initially comprised in a liquid sample, the method comprising
concentrating said macromolecule or said agglomerate within a layer using the method according to claim 1, then
replacing said liquid sample, within said layer, with a liquid comprising amplification agents, and then
amplification amplifying by means of said agents.

17. The method according to claim 16, wherein the macromolecule is a DNA.

18. The method according to claim 16, wherein the agglomerate of molecules is a prion.

19. The method according to claim 5, wherein said molecule is a surfactant molecule.
